(19) United States
(12) Patent Application Publication
(10) Pub. No.: US 2017/0202691 A1
(43) Pub. Date: Jul. 20, 2017

(54) SENSOR-STENTS

(71) Applicants: Jeffrey LABELLE, Tempe, AZ (US); Aaron MEIDINGER, Gilbert, AZ (US)

(72) Inventors: Jeffrey LABELLE, Tempe, AZ (US); Aaron MEIDINGER, Gilbert, AZ (US)

(73) Assignee: ARIZONA BOARD OF REGENTS ON BEHALF OF ARIZONA STATE UNIVERSITY, Scottsdale, AZ (US)

(21) Appl. No.: 15/313,826

(22) PCT Filed: May 27, 2015
(86) PCT No.: PCT/US15/32584

§ 371 Date: Nov. 23, 2016

Related U.S. Application Data
(60) Provisional application No. 62/004,630, filed on May 29, 2014.

(51) Int. Cl. A61F 2/915; A61B 5/02

(52) U.S. Cl. CPC A61F 2/915; A61B 5/02007; A61F 2002/91575; A61F 2240/002; A61F 2250/0002; A61B 2562/0261

(57) ABSTRACT

Stents adapted to allow for monitoring an environment into which they have been inserted in a body, as well as methods of making and using such stents and systems involving such stents. Such stents allow for the detection and treatment of side effects and deleterious results of stent insertion. These stent are makeable by processes and methods involving three dimensional printing.

### CROSS REFERENCE TO RELATED APPLICATIONS

This application claims benefit to U.S. Provisional Patent Application No. 62/004,630 titled “SENSOR-STENTS” filed May 29, 2014. The entire contents of the referenced patent application are incorporated into the present application by reference

### BACKGROUND OF THE INVENTION

1. Field of the Invention

The present invention relates generally to the field of stents for placement in a subject's body. More particularly, it concerns stents that allow for monitoring of an environment into which a stent has been placed to allow for determination of potential complications from the placement of the stent.

2. Description of Related Art

Heart attacks are a leading cause of death in the United States. The most common cause of heart attack occurs when plaque builds up in the coronary artery and ruptures, providing a surface for blood to clot, which grows to partially block the artery. Even if the plaque does not rupture, it still reduces the flow of blood and oxygen to the heart, increasing risk of heart attack. When proper diet and medication fail to prevent or reverse the build-up of this plaque, more drastic measures must be taken. Early methods for treating this condition involved complex and risky surgeries.

Angioplasty is a procedure in which a thin catheter with a balloon at the end is inserted into the passageway via a small incision, and the balloon is inflated to compress the plaque against the vessel walls and expand the vessel itself, widening the passage and increasing the flow of blood. Angioplasty alone was an imperfect solution with a high incidence of re-intervention. The most common problem with angioplasty is restenosis, which is defined as a reduction in vessel diameter of more than 50% in response to the stress and damage on the vessel walls as a result of the procedure. The effect can be acute, sudden, and dangerous.

The dangers of restenosis lead to the increased use of stents, which are typically small metal mesh cylinders. Stents are now regularly implanted in a modified form of angioplasty. The metal mesh cylinder is compressed around the end of the catheter, and when the balloon is expanded the stent expands too. The balloon is deflated and the stent remains behind to hold the vessel open. Angioplasty with stenting results in a great decrease in restenosis rates as compared to plain angioplasty.

stents can cause their own set of problems. The stress on the blood vessel caused by typical angioplasty is more focused due to the fact that the stent mesh is in contact with the vessel walls, not the balloon. This more focused injury can lead to in-stent restenosis, where the damage to the vessel is worse than with traditional angioplasty. In order to reduce this restenosis problem, drugs were developed to mitigate the reaction of the vessel. These drug eluting stents have been shown to have less than a 5% rate of in-stent restenosis.

With in-stent restenosis greatly reduced by the widespread usage of drug eluting stents, the last major problem caused by stents is stent thrombosis. stent thrombosis occurs when a blood clot forms on the foreign surface of the stent in the blood stream. While stent thrombosis has a relatively low incidence of occurrence (2% or less), the mortality rate of those occurrences could be as high as 45%. The mortality rate is high because the problem can develop extremely quickly and, similar to plaque rupture, cause a heart attack. In order to detect stent thrombosis in formation before it becomes a problem, it would be necessary to constantly perform expensive tests and scans. With such a low incidence, it is not feasible to mandate that every stent patient repeatedly have these scans done to check for thrombosis formation. However, with such a severe risk, a solution must be found.

In summary, there are significant difficulties in detecting and treating side effects and deleterious results of stent insertion, and further improvements in the area would benefit patients and providers.

### SUMMARY OF THE INVENTION

The present invention relates to a stent adapted to allow for monitoring an environment into which it has been inserted in a body as well as methods of making and using such stents and systems involving such stents. Such stents allow for the detection and treatment of side effects and deleterious results of stent insertion.

Typically, the stents of the present invention are comprised of a resistive material and a conductive material. In some embodiments, the resistive material is a plastic and/or a composite of plastic and other components such as, but not limited, to a metal and/or ceramic. Further, in some cases the conductive material is and/or comprises a plastic, a metal, a ceramic, or a composite. In some existing embodiments, a conductive filament for a three-dimensional printer was used. Additional, conductive materials may comprise a plastic with at least one conductive additive. In some embodiments, the stent further comprises an electo-insulative covering. The electro-insulative covering may be any known to those of skill. Typically, the stents will be formed of biocompatible materials and/or coated with biocompatible materials.

In some embodiments, the stents may be made from at least one material selected from the group consisting of polypeptides, polydepsipeptides, nylon copolymides, aliphatic polyesters, polydihydropyrans, polyphosphazenes, polyorthoesters, polycyanoacrylates, and their derivatives. Some of these may be biodegradable after a period of time in the body. Bioactive agents may also be incorporated into the material. These agents may be selected from the group consisting of heparin, hirudin, warfarin, ticlopidine, dipyridamole, GPIIb/IIIa receptor blockers, thromboxane inhibitors, serotonin antagonists, prostanoids, calcium channel blockers, PDGF antagonists, ACE inhibitors, angiopeptin, enoxapalin, colchicine, steroids, non-steroidal anti-inflammatory drugs, VEGF, adenovirus, enzymes, sterol, hydroxylase, antisense sequences, fish oils, HMG, Co-A reductase inhibitors, ibutilide fumarate, adenylcyclase, growth factors, nitric oxide, proteins, peptides and carbohydrates. Of course, those of ordinary skill will know of many alternatives for materials and agents.

In stents of some embodiments of the invention, at least one of the restive material and the conductive material is a material that can be formed by a three dimensional printing process. In more specific embodiments, the three dimensional printing process comprises fused deposition modeling, selective laser sintering, selective laser annealing, selective heat sintering, stereolithography, digital light processing, electron beam melting, electron beam freeform fabrication, centrifugal spin casting and/or direct metal laser sintering.

The stents of the invention are adapted to monitor the environment by measuring electrical resistance if the stent is deformed during use. In specific embodiments, the stent is adapted to transmit data about the electrical resistance of the stent to a receiver outside of the body during use. Such transmission may, in some cases, be wireless, with the stent comprising or being coupled to a transmitter in the body. Some particular embodiments of the stent can be adapted to generate energy for measuring resistance and/or transmitting data from physical movements of or in the body.

In some embodiments, the stent acts as a sensor in the manner of a strain gauge. A strain gauge is a section of resistive material bonded to the surface of a body of known dimensions. When a force acts on the body, the resulting stress produces a predictable strain, and the body is deformed. The resistive material itself deforms along with the surface of the body to which it is attached. The resistive material is oriented such that mechanical stress in the proper direction deforms the strain gauge. When this happens, the strain gauge is acting as an electrical resistor, which follows this equation:

$$R = ρ \frac{L}{A}$$

where R is the resistance, ρ is the resistivity, L is the length of the current path, and A is the cross-sectional area. A deformation in the direction of L increases L and decreases A, causing the resistance to increase. If the material properties and dimensions of the body are known, the resistance of the strain gauge can be measured to calculate the force applied to the body. In the current invention, the stents have known and selected material properties and dimensions that allow them to function as a strain gauge in the body.

The stents may be adapted to determine a physiological change at a location of insertion in the body during use. For example, in embodiments where the stent is configured to be placed in a human artery or vein, the physiological change can be restenosis, stent thrombosis, or a symptom of either of these. Stents can also be used to divert blood flow from an aneurysm (typically in the abdominal aorta or in the brain), where they are used to divert blood from an aortal tear. Aneurysm stents usually have a type of fabric to enable them to divert blood more effectively.

The invention also contemplates methods of using these stents, which method generally comprises: obtaining a stent of claim 1, inserting the stent into a subject's body, and monitoring an environment in the body into which the stent has been inserted. In many cases, the stent is placed in a human artery or vein. The method of monitoring will typically comprise monitoring electrical resistance in the stent. In some embodiments, the monitoring is via wireless transmission of information from the stent to a receiver outside of the body. Such monitoring can allow for detection of a physiological change at a location of insertion in the body. In the instance where the stent is in a vein or artery, the physiological change may be indicative of restenosis, stent thrombosis, aneurism, or a symptom of any of these.

Such methods may also comprise treating the subject in response to a detected physiological change. For example the treatment may comprise restenosis treatments including but not limited to brachytherapy, intracoronary radiation, angioplasty, additional stenting inserted inside the original, inclusion of a drug-eluting stent. Treatments of thrombosis may comprise an aspiration thrombectomy and/or placement of a new stent. Of course skilled medical practitioners will know appropriate treatments in appropriate circumstances.

The stent may be inserted in a body by means appropriate to their design. One such method would be to fit the collapsed stent over an inflatable element of a balloon catheter and expand the balloon to force the stent into contact with a vein or artery. As the balloon is inflated, the vessel is compressed in a direction generally perpendicular to the wall of the vessel which, consequently, dilates the vessel to facilitate blood flow.

The methods of using the stent may further comprise producing the stent using a three dimensional printing process according to dimensions of the location in the body into which the stent is to be inserted. In this manner, stents may be custom produced for a specific site in the body and/or for the needs of a specific patient.

The stents can be made in a manner that allows them to expand into a final form after insertion into the body. For example, a flat stent may be rolled prior to insertion, allowing for the stent to unroll to form a channel inside a lumen of the body after insertion. Such a lumen could, for example, be an artery or vein. Alternatively, they may be substantially cylindrical prior to insertion, but inserted in a compressed manner.

The invention also contemplates methods of producing the stents comprising: obtaining a resistive material and using a process comprising three dimensional printing to form a stent comprising the resistive material. These methods may further comprise obtaining a conductive material and using a process comprising three dimensional printing to form a stent comprising the conductive material. The methods may further comprise forming an electo-insulative covering on the stent. The materials used can be any of the ones discussed above and/or known to those of skill in the art at the time of making of the stents.

In some specific embodiments, the three dimensional printing process comprises fused deposition modeling, selective laser sintering, selective heat sintering, selective laser annealing, stereolithography, digital light processing, electron beam melting, electron beam freeform fabrication, centrifugal spin casting, and/or direct metal laser sintering.

The methods may comprise connecting the stent to a wireless transmitter adapted to wirelessly transmit data about the electrical resistance of the stent to a receiver outside of the body during use and/or incorporating such a transmitter into the stent. A power source may be incorporated into the stent and/or in conjunction with the transmitter.

In some embodiments, the three dimensional printing produces a substantially flat structure. This flat structure may be rolled into a cylinder or spiral prior to insertion. In some embodiments, there is a step of fusing edges of the flat structure to form a cylinder during insertion. In other embodiments, the three dimensional printing process produces a structure that is not substantially flat. In some embodiments, the method is further defined as a method of producing a stent using a three dimensional printing process according to dimensions of the location in the body into which the stent is to be inserted. In this manner, stents may be custom produced for a specific site in the body and/or for the needs of a specific patient. Alternatively, there can be standard sizes from which a to select for a given procedure.

The invention also contemplates systems comprising a stent as described above and a receiver.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows various prototype designs for the stents of the invention.

FIG. 2 is a 3D model of finalized stent design with photograph of actual stent; the stent has a 0.3 mm thickness and a 0.25″ diameter.

FIGS. 3A and 3B shows the result of a study in which stents were compressed and their resistances measured.

FIGS. 4A and 4B show graphs of pressure over time and voltage over time from the compressed stents.

FIG. 5 is a calibration curve for the stent maps voltage to pressure.

### DESCRIPTION OF ILLUSTRATIVE EMBODIMENTS

The invention and the various features and advantageous details are explained more fully with reference to the non-limiting embodiments that are illustrated in the accompanying drawings and detailed in the following description. Descriptions of well-known starting materials, processing techniques, components, and equipment are omitted so as not to unnecessarily obscure the invention in detail. It should be understood, however, that the detailed description and the specific examples, while indicating embodiments of the invention, are given by way of illustration only and not by way of limitation. Various substitutions, modifications, additions, and/or rearrangements within the spirit and/or scope of the underlying inventive concept will become apparent to those skilled in the art from this disclosure.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Cardiac stents of the invention were designed such that the stents and the surrounding tissue can be continuously monitored for any sign of restenosis or thrombosis. These problems associated with stents can be characterized by a constricting of blood-flow in the affected vessel. Using the basic fluid mechanics definition of mass flow rate,

\dot{m}=ρvA

where ρ is the density of the fluid, v is the velocity, and A is the cross-sectional area, and assuming constant flow rate and density, equation 1 relates cross-sectional area to velocity.
As the area is reduced, the velocity must increase. Bernoulli's equation,

$$\frac{{p}_{1}}{γ} + \frac{{v}_{1}^{2}}{2 g} + {h}_{1} = \frac{{p}_{2}}{γ} + \frac{{v}_{2}^{2}}{2 g} + {h}_{2} + Σ {h}_{1}$$

assuming gravity, energy, density, and height are constant simplifies to

$$\frac{{p}_{1}}{γ} + \frac{{v}_{1}^{2}}{2 g} = \frac{{p}_{2}}{γ} + \frac{{v}_{2}^{2}}{2 g}$$

which relates velocity of a flow to pressure. The assumptions of constant mass flow rate, zero energy input, and constant height are all valid along a small length of blood vessel. Therefore, a constriction in area under these conditions will result in an increase in velocity and a decrease in pressure. In order to measure the local pressure decrease along a stent, the inventors designed a stent that is itself a sensor by making the sensor that could provide data in the same manner as a strain gauge.

Using a conductive 3D-printer filament in conjunction with a Makerbot Replicator Dual® 3D-printer and white ABS plastic filament, enabled preliminary studies to prove that strain gauges could be produced via three dimensional printing (data not shown). A conductive plastic filament was used as well in some studies.

### Example 1

Stents were printed as a flat mesh. Because 3D-printing progresses by layers, each layer can be a continuous extrusion of plastic, but in between layers the strength is diminished as the first layer cools. Testing of a variety of prototype stents optimized a stent of strength, flexibility, and size.

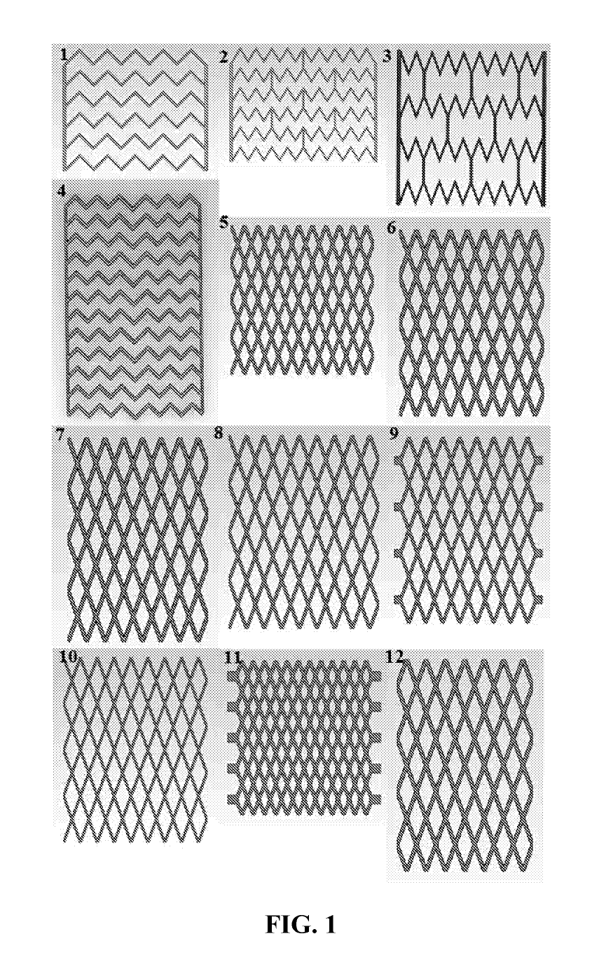

FIG. 1 shows a variety of stent designs that were created using Solidworks® with white ABS. Stent designs 1-4 were focused on achieving maximum flexibility. While their flexibility was high, their stability was poor. When folded into a cylindrical shape, the diameter was inconsistent. Stent 5 sacrificed some of that flexibility and complexity to achieve simplicity and good stability. Stent designs 6-11 experimented with varying sizes of diamond, thickness of the struts, and vertical thickness of the horizontal stent. These tests were all focused on optimizing flexibility and strength. Bonding the stents into cylindrical form involved using acetone to fuse the ends to one another.

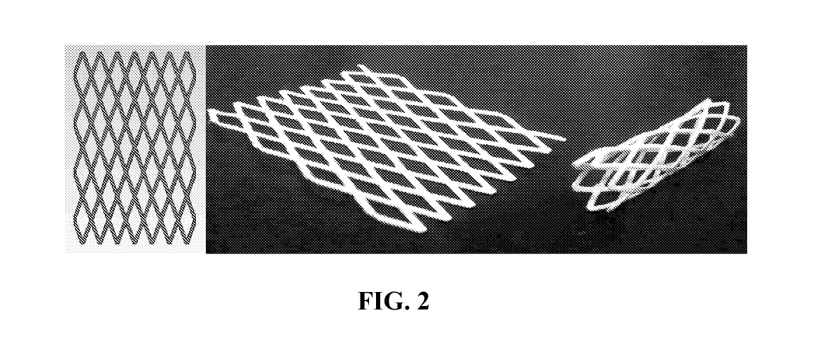

The final design, stent 12, is capable of folding to an effective diameter of 0.25″. FIG. 2 shows the design, the flat printed stent, and the stent after bonding into a cylindrical form. The stent is three layers thick, with layer height 0.1 mm each. The stents were printed at 230° C. with a heated printing platform at 110° C., which encourages strong bonding between layers. Three layers was determined to be the best for a blend of flexibility and strength with regard to the particular application. The design is on the same order of magnitude as stents currently used.

### Example 2

To confirm that the stents produced in Example 1 would operate as strain gauges, they were subjected to the same type of stress to which they would be subjected to when implanted.

A set of analog calipers were coated with an insulating tape and used to compress the stent to precise diameters. A digital multi-meter was used to directly measure resistance, not voltage. The diameter and resistance value were recorded and graphed. The voltage across the stent was measured periodically, both while diameter was increasing and decreasing in order to detect any permanent damage as evidenced by a baseline shift.

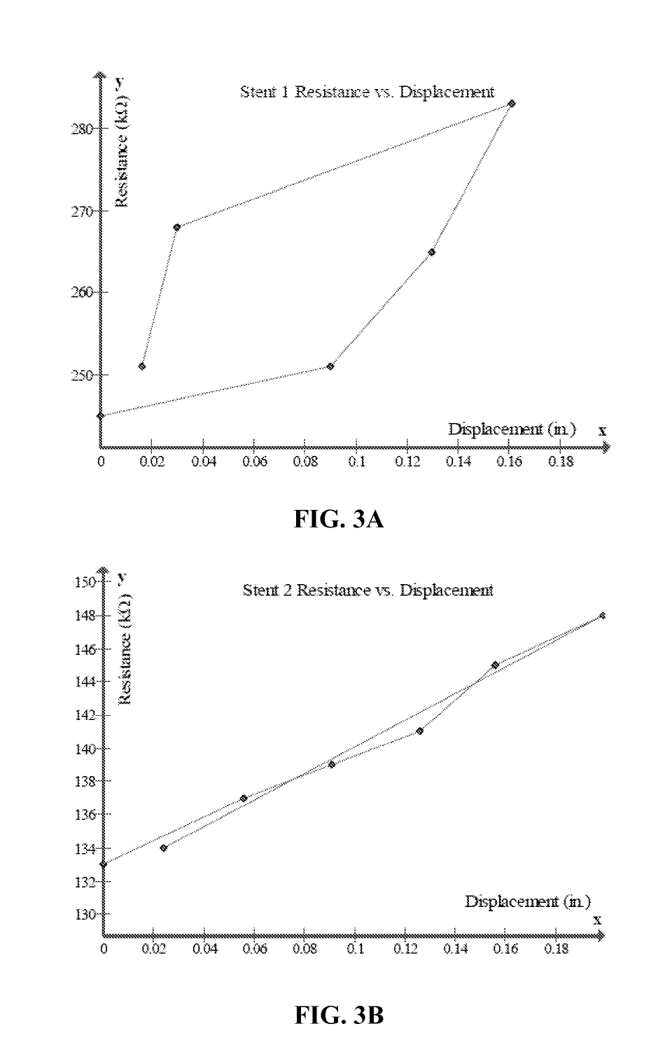

FIGS. 3A and 3B show the results of this test. As the cylindrical stents compressed into an oval, the relationship between displacement and resistance was positive, and the resistance of the sensor at the end of the experiment is extremely close to what it was at the beginning. This indicates that the stents can be used to collect information about pressures in the stent, because conditions that stop the flow of blood through the passageway by narrowing the vessel, decrease the area. Therefore, any pressure decrease relates to an area decrease, indicating a problem such as restenosis or thrombosis. This enables one to monitor for such directly in real time and in vivo.

### Example 3

To confirm that the stents could detect changes in pulsatile flow, like those found in the blood stream, a further study was conducted with a stent employing actual fluid flow, driven by a peristaltic pump to simulate blood flow conditions.

A 40 MΩ test stent was connected in series with an 18 MΩ control stent. The peristaltic pump created a sinusoidal pressure that was measured using two sensors connected to an Iworx® 214 station, and a data-logging digital multi-meter measured the voltage drop across the test stent resulting from the applied 1V DC.

Specifically, two diameters of blood vessel mimicking vascular graft were combined with one stent to create a test sample. The untreated stent was rolled up and placed inside the larger diameter graft. The thinner diameter was able to slide into the center of the stent with minimal friction. The outer graft material was used to make sure the stent made contact with the inner tube, ensuring it would experience the same pressure changes. However, unlike a normal stent, this stent was actually mounted outside the vessel that would receive the flow. This was necessary because otherwise the experiment would require an insulating coating to keep water out of the electrical circuit. The test sample was connected to a system powered by a peristaltic pump. This pump creates a pulsatile flow similar to the way a heart does, rather than a traditional pump with constant flow. As a result of the placement of the stent and the tightness of the fit, the sensor only experiences the peaks of the pressure and not the valleys.

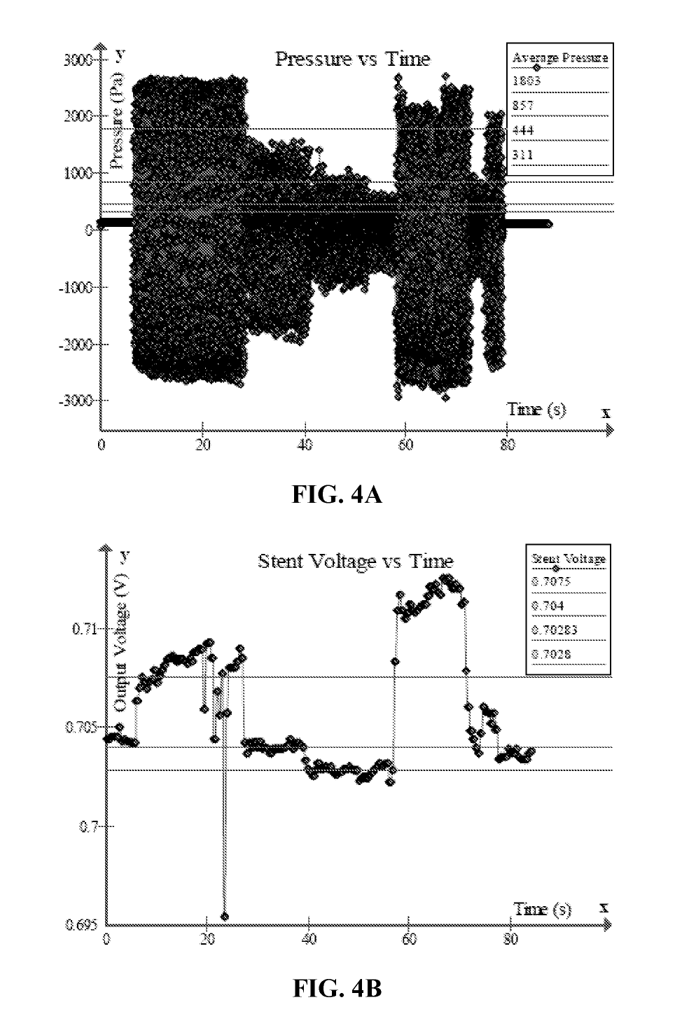

To take measurements, both a data-logging digital multi-meter and an Iworx® 214 station were used. As before, the stent test specimen was connected in series with another stent to create a voltage divider. A potential of 1V was supplied, and the resistances of the test stent and the series stent was 40 MΩ and 18 MΩ, respectively. The digital multi-meter measured the voltage drop across the test specimen twice every second, and the Iworx® station was connected to two pressure sensors on either side of the specimen. FIGS. 4A and 4B show the data for one such test.

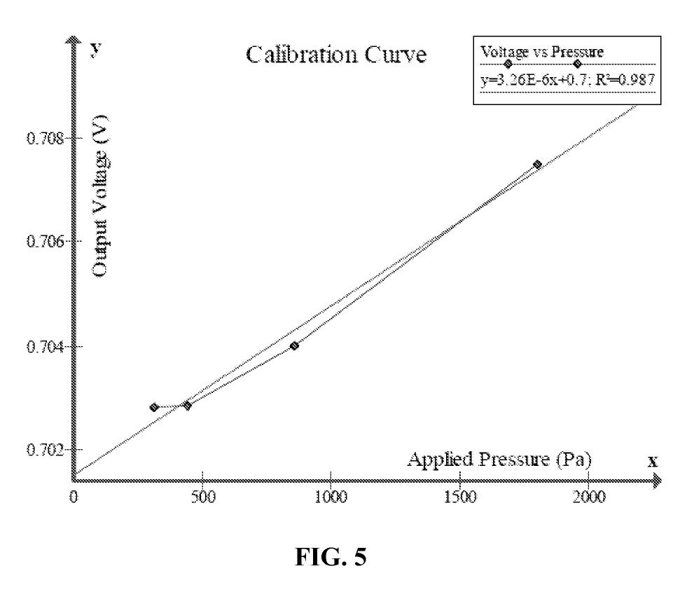

FIG. 5 displays the graphs of the pressure versus time data as well as stent voltage versus time. Because the pressure graph is sinusoidal, the root mean square of the pressure was taken over the sections corresponding to the various pump settings. These values were correlated to the average voltage readings from the stent in order to create a sensor calibration curve. According to the calibration curve, the sensor output in this current configuration is 3.2*10-6 volts per RMS gauge pressure in pascals. This graph exemplifies that the output of the sensor changes with the input (pressure) on the sensor. From the equation discussed above, one can deduce that pressure output is directly related to the flow speed of blood, which is related to the area of the passage way. Problems that stop the flow of blood through the passageway by narrowing the vessel, decreasing the area. So a pressure decrease relates to an area decrease, indicating a problem such as restenosis or thrombosis.

All of the embodiments disclosed and claimed herein can be made and executed without undue experimentation in light of the present disclosure. While the stents and methods of this invention have been described in terms of preferred embodiments, it will be apparent to those of skill in the art that variations may be applied to the them and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Grove E. C. L and Kristensen S. D., “Stent thrombosis: definitions, mechanisms and prevention.” European Society of Cardiology, 8 May 2007. www.escardio.org/communities/councils/ccp/e-journal/volume5/Pages/vol5n32.aspx#.Up5FW8S1xcY. 3 Dec. 2013.
Huda Hamid and John Coltart, “‘Miracle stents’—a future without restensosis”. McGill Journal of Medicine. National Center for Biotechnology Information, July 2007. www.ncbi.nlm.nih.gov/pmc/articles/PMC2323487/. 3 Dec. 2013.
“What Is Coronary Angioplasty?” National Heart, Lung, and Blood Institute, 1 Feb. 2012. http://www.nhlbi.nih.gov/health/health-topics/topics/angioplasty/. 3 Dec. 2013.

1.-38. (canceled)

39. A stent adapted to allow for monitoring an environment into which it has been inserted in a body.

40. The stent of claim 39, further defined as comprised of a resistive material and a conductive material.

41. The stent of claim 40, wherein the resistive material comprises a plastic, metal, ceramic, or composite.

42. The stent of claim 40, wherein the conductive material comprises a plastic, metal, ceramic, or composite.

43. The stent of claim 40, wherein at least one of the restive material and the conductive material are materials that can be formed by a three dimensional printing process.

44. The stent of claim 40, wherein the three dimensional printing process comprises fused deposition modeling, selective laser sintering, selective laser annealing, selective heat sintering, stereolithography, digital light processing, electron beam melting, electron beam freeform fabrication, and/or direct metal laser sintering.

45. The stent of claim 40, further comprising an electo-insulative covering.

46. The stent of claim 39, further defined as adapted to monitor the environment by measuring electrical resistance if the stent is deformed during use.

47. The stent of claim 46, wherein the stent is adapted to wirelessly transmit data about the electrical resistance of the stent to a receiver outside of the body during use.

48. The stent of claim 46, further defined as adapted to generate energy for measuring resistance and/or transmitting data from physical movements of or in the body.

49. The stent of claim 46, further defined as adapted to determine a physiological change at a location of insertion in the body during use.

50. The stent of claim 49, wherein the physiological change is restenosis, stent thrombosis, aneurysm, or an aortal tear or a symptom of any of these.

51. The stent of claim 39, further defined as being comprised of a material that will dissolve at a desired time after insertion in the body.

52. The stent of claim 39, further defined as configured to be placed in a human artery or vein.

53. A method comprising:
obtaining a stent of claim 39;
inserting the stent into a subject's body; and
monitoring an environment in the body into which the stent has been inserted.

54. The method of claim 53, wherein the stent is placed in a human artery or vein.

55. The method of claim 53, wherein monitoring the environment comprises monitoring electrical resistance in the stent.

56. The method of claim 55, wherein the monitoring is via wireless transmission of information from the stent to a receiver outside of the body.

57. The method of claim 53, further defined as a method of detecting a physiological change at a location of insertion in the body.

58. The method of claim 57, wherein the physiological change is indicative of restenosis, stent thrombosis, aneurysm, or an aortal tear or a symptom of either of these.

59. The method of claim 57, further comprising treating the subject in response to a detected physiological change.

60. The method of claim 21, wherein the treatment comprises angioplasty, stent implantation, brachytherapy, intracoronary radiation, and/or aspiration thrombectomy.

61. The method of claim 53, further comprising producing the stent using a three dimensional printing process according to dimensions of the location in the body into which the stent is to be inserted.

62. The method of claim 53, where the stent is rolled prior to insertion.

63. The method of claim 53, wherein the stent is substantially cylindrical prior to insertion.

64. The method of claim 53, wherein the stent is adapted to expand after insertion.

65. A method of producing a stent of claim 39, comprising:
obtaining a resistive material; and
using a process comprising three dimensional printing to form a stent comprising the resistive material.

66. The method of claim 65, wherein the resistive material is a plastic, metal, ceramic, or composite.

67. The method of claim 65, further comprising
obtaining a conductive material; and
using a process comprising three dimensional printing to form a stent comprising the conductive material.

68. The method of claim 67, wherein the conductive material is a plastic, metal, ceramic, or composite.

69. The method of claim 65, wherein the three dimensional printing process comprises fused deposition modeling, selective laser sintering, selective heat sintering, stereolithography, digital light processing, electron beam melting, electron beam freeform fabrication, and/or direct metal laser sintering.

70. The method of claim 65, further comprising forming an electo-insulative covering on the stent.

71. The method of claim 65, further comprising connecting the stent to a wireless transmitter adapted to wirelessly transmit data about the electrical resistance of the stent to a receiver outside of the body during use.

72. The method of claim 65, wherein the three dimensional printing produces a substantially flat structure.

73. The method of claim 72, further comprising rolling the flat structure into a cylinder or spiral prior to insertion.

74. The method of claim 73, further comprising fusing edges of the flat structure to form a cylinder during insertion.

75. The method of claim 65, wherein the three dimensional printing process produces a structure that is not substantially flat.

76. A system comprising a stent of claim 39 and a wireless receiver.
